SEQUENCE LISTING (18 sequences)

SEQ ID NO: 1 - DNA, 20 bp, Artificial Sequence
  forward primer sequence of 18S
gtaacccgttgaaccccatt

SEQ ID NO: 2 - DNA, 19 bp, Artificial Sequence
  forward primer sequence of 18S
gtacccgcacttgcacaac

SEQ ID NO: 3 - DNA, 19 bp, Artificial Sequence
  forward primer sequence of SOX9
gtacccgcacttgcacaac

SEQ ID NO: 4 - DNA, 21 bp, Artificial Sequence
  forward primer sequence of SOX9
tctcgctctcgttcagaagtc

SEQ ID NO: 5 - DNA, 19 bp, Artificial Sequence
  forward primer sequence of collagen2a
gggagtaatgcaaggacca

SEQ ID NO: 6 - DNA, 20 bp, Artificial Sequence
  forward primer sequence of collagen2a
atcatcaccaggctttccag

SEQ ID NO: 7 - DNA, 18 bp, Artificial Sequence
  forward primer sequence of aggrecan
gcctgcgctccaatgact

SEQ ID NO: 8 - DNA, 21 bp, Artificial Sequence
  forward primer sequence of aggrecan
atggaacacgatgcctttcac

SEQ ID NO: 9 - DNA, 21 bp, Artificial Sequence
  forward primer sequence of collagen a1
cccctggaaagaatggagatg

SEQ ID NO: 10 - DNA, 21 bp, Artificial Sequence
  forward primer sequence of collagen a1
tccaaaccactgaaacctctg

SEQ ID NO: 11 - DNA, 21 bp, Artificial Sequence
  forward primer sequence of MMP13
tcaccaattcctgggaagtct

SEQ ID NO: 12 - DNA, 21 bp, Artificial Sequence
  forward primer sequence of MMP13
tcaccaattcctgggaagtct

SEQ ID NO: 13 - DNA, 20 bp, Artificial Sequence
  forward primer sequence of collagen 10
acgctgaacgataccaaatg

SEQ ID NO: 14 - DNA, 27 bp, Artificial Sequence
  forward primer sequence of collagen 10
tgctatacctttactctttatggtgta

SEQ ID NO: 15 - DNA, 20 bp, Artificial Sequence
  forward primer sequence of p53
ggcccacttcaccgtactaa

SEQ ID NO: 16 - DNA, 20 bp, Artificial Sequence
  forward primer sequence of p53
gtggtttcaaggccagatgt

SEQ ID NO: 17 - DNA, 20 bp, Artificial Sequence
  forward primer sequence of BAX
tttgcttcagggtttcatcc

SEQ ID NO: 18 - DNA, 20 bp, Artificial Sequence
  forward primer sequence of BAX
cagttgaagttgccgtcaga

(19) United States
(12) Patent Application Publication
(10) Pub. No.: US 2022/0296649 A1
(43) Pub. Date: Sep. 22, 2022

(54) METHOD FOR PREPARING MATRILIN-3 PRETREATED STEM CELL SPEROIDS, AND COMPOSITION, DERIVED THEREFROM, FOR PREVENTING OR TREATING CARTILAGE DISEASES

(71) Applicant: SUNGKWANG MEDICAL FOUNDATION, Seoul (KR)

(72) Inventors: In Bo HAN, Gyeonggi-do (KR); Hye Min CHOI, Gyeonggi-do (KR)

(73) Assignee: SUNGKWANG MEDICAL FOUNDATION, Seoul (KR)

(21) Appl. No.: 17/633,960

(22) PCT Filed: Aug. 11, 2020
(86) PCT No.: PCT/KR2020/010629

§ 371 Date: Feb. 9, 2022

(30) Foreign Application Priority Data
Aug. 12, 2019 (KR) 10-2019-0098344

(51) Int. Cl. A61K 35/35; C12N 5/077; A61P 19/02

(52) U.S. Cl. CPC A61K 35/35; C12N 5/0653; A61P 19/02; C12N 2501/998; C12N 2506/1384; C12N 2513/00

(57) ABSTRACT

Provided are a method of preparing a spheroid of stem cells and a composition including the spheroid prepared by the method, the method including: culturing stem cells in a medium supplemented with matrilin-3 protein; and performing 3D cell culture on the cultured stem cells in the medium. The composition disclosed herein has effects of preventing or treating cartilage disease. In detail, the composition may be able to further promote cartilage differentiation of adult stem cells and reduce dedifferentiation and hypertrophy that may occur during cartilage regeneration, thereby providing a more effective cartilage tissue regeneration method.

### TECHNICAL FIELD

The present disclosure relates to a method of preparing a matrilin-3-primed stem cell spheroid and a composition for treating cartilage disease derived thereby.

### BACKGROUND ART

Treatments for degenerative cartilage disease have been attracting attention due to the aging of the global population. Particularly in South Korea, the incidence of cartilage disease is reported to be remarkably increasing even in the age group of 50s or less due to sedentary lifestyles and bad living habits. In cartilage tissues, the aging causes reduction in cartilage thickness and the number of chondrocytes and changes in matrix components and cell functions. However, cartilage is devoid of blood vessels, nerves, and lymphatics, and thus is characterized by being unable to regenerate itself after injury. As such, in accordance with the increasing incidence of degenerative cartilage disease, attention is further focused on the development of treatment.

To treat lumbar pain from degenerative cartilage disease, conservative treatments, such as drugs and physical therapies, have been mainly used in the past. When such conservative treatments were ineffective, surgical treatment was also being considered. In the latter case, the surgical treatment may have an effect on significant pain reduction within a short period of time. However, in the long term, the surgical treatment may worsen degenerative changes and cause spinal instability, resulting in more severe lumbar pain. That is, there is a limitation that the existing surgical treatment is not a method to fundamentally treat degenerative cartilage disease.

To overcome such a limitation, research on various biological treatments for regeneration of degenerative intervertebral discs has been continued. In detail, most attempts have been to supplement deficient growth factors. Growth factors, such as transforming growth factor (TGF)β, insulin like growth factor-1, and bone morphogenic protein-2, may be injected into cartilage tissues including modified intervertebral discs to stimulate matrix production. However, the injected growth factors are destroyed over time by in vivo degradable proteins in vivo, requiring continuous injection of the growth factors. Also, since the growth factors are available from other animals, there is a limitation that expensive production costs are incurred. To overcome such a limitation, research has been conducted on a treatment method in which growth factors are expressed all the time through genetic manipulation. However, many limitations for immediate clinical application are present.

As a treatment method to overcome such limitations, treatment using adult stem cells is attracting attention. The adult stem cells may overcome canceration and ethical issues that are common with embryonic stem cells, and may be differentiated into various cells including adipocytes, osteoblasts, chondrocytes, cardiac cells, muscle cells, and nerve cells. In this regard, the adult stem cells have sufficient potency as cell therapy products, but it is true that there are still many limitations. For example, when monolayer culture of the stem cells is performed for a long period of time to obtain a sufficient number of cells required for a surgical procedure, the stem cells may have reduced phenotype and dedifferentiation may occur.

Therefore, the present disclosure is to provide a more fundamental treatment based on studies on a 3D cell culture method of the stem cells during tissue regeneration using stem the cells that are widely used for the tissue regeneration.

### DESCRIPTION OF EMBODIMENTS

### Technical Problem

An object of the present disclosure is to provide a method of preparing a spheroid of stem cells, the method including: culturing stem cells in a medium supplemented with matrilin-3 (MATN-3) protein; and performing 3D cell culture on the cultured stem cells in the medium.

Another object of the present disclosure is to provide a spheroid prepared by the method.

Another object of the present disclosure is to provide a composition for treating cartilage disease, including the spheroid prepared by the method.

### Solution to Problem

An aspect of the present disclosure provides a method of preparing a spheroid of stem cells, the method including: culturing stem cells in a medium supplemented with matrilin-3 (MATN-3) protein; and performing 3D cell culture on the cultured stem cells in the medium.

The term “matrilin-3 (MATN-3) protein” as used herein refers to a matrilin-based protein, which is one of proteins constituting the von Willebrand factor A domain, and may be present in the extracellular matrix of cartilage.

The MATN-3 protein may be expressed by a MATN-3 gene. The MATN-3 protein may be expressed by a MATA-3 gene particularly derived from a mouse or a human. More particularly, the MATN-3 protein may be expressed by a human-derived MATA-3 gene.

The term “stem cells” as used herein refers to cells having ability to differentiate into various types of body tissues. Also, the stem cells refer to cells that can differentiate into various tissue cells when setting conditions in an undifferentiated state.

In an embodiment of the present disclosure, the stem cells may be adult stem cells, but are not limited thereto. The adult stem cells refer to stem cells that appear in the stage in which each organ of an embryo is formed through a differentiation process, or in the adult stage. In this regard, the adult stem cells refer to undifferentiated cells capable of regenerating without limitation to form cells specialized for tissues and organs. The stem cells used in the present disclosure may be mesenchymal stem cells particularly derived from bone marrow, embryo, umbilical cord blood, and other various adult tissues including placenta, alveolar bone, muscle, fat, and nervous tissue. More particularly, the stem cells may be adipose-derived mesenchymal stem cells. To differentiation into chondrocytes, bone marrow-derived mesenchymal stem cells have been mainly used in the past, and adipose-derived mesenchymal stem cells have been pointed out regarding poor differentiation potency into chondrocytes compared to bone marrow-derived mesenchymal stem cells. In an embodiment of the present disclosure, by confirming that MATN-3 promotes differentiation potency of adipose-derived stem cells into chondrocytes, disadvantages of the adipose-derived mesenchymal stem cells with respect to the differentiation into chondrocytes may be overcome. Any cell may be used regardless of where it originates from, but more particularly, cells used herein may be derived from mammals including humans, mice, rats, rabbits, dogs, cows, horses, pigs, sheep, cats, monkeys, and goats. The stem cells may be stem cells of passages 1 to 100. In particular, the stem cells may be adult stem cells of passages 1 to 30, or may be embryonic stem cells of passages 1 to 100. Also, more particularly, the stem cells may be human-derived stem cells. Also, more particularly, the stem cells may be adipose-derived mesenchymal stem cells.

The term “mesenchymal stem cell (MSC)” as used herein refers to a stem cell having multipotency and self-renewability, and may be stem cells that can differentiate into various cells, such as adipocytes, chondrocytes, osteocytes, and the like.

The term “differentiation” as used herein refers to a phenomenon in which a cell structure or a cell function is specialized to each other during division, proliferation, and growth of cells. That is, differentiation refers to a change in form or function of cells, tissues, and the like of living organisms to perform a given task thereof.

The term “culture” as used herein refers to a cell culture process in which isolated cells are cultured in a medium. As the medium, any medium generally used for culturing stem cells may be used. For example, the medium may be minimum essential medium alpha (MEM-alpha), mesenchymal stem cell growth medium (MSCGM), Dulbecco's modified Eagle's medium (DMEM), and the like. The medium may be supplemented with glucose, insulin, selenium, transferrin, and vascular endothelial growth factor (VEGF).

In an embodiment of the present disclosure, the culture may be 3D cell culture.

The 3D cell culture is not a conventional 2D culture method using a medium, but a culture method of culturing cells in a 3D manner. The 3D cell culture may refer to a cell culture model that allows cells to grow in all dimensions or interact with the surrounding environment by artificially creating an environment, which is similar to a living body, in vitro. When the 3D cell culture is performed in a 3D space consisting of extracellular matrix components, cells are supplied with nutrients, oxygen, and drugs through diffusion gradient and permeation, so as to provide an environment similar to a living body. In this regard, the 3D cell culture may be characterized by 3D contact interactions between cells, paracrine signaling by diffusion of cell secretions, and the like. In comparison with the existing culture methods, the 3D cell culture may be characterized by frequent heterogeneous exposure, feasible cell-to-cell communication, and a high differentiation rate.

The 3D cell culture may be selected from the group consisting of pellet culture, static suspension culture, spinner/rotational chamber culture, nano/micro pattern culture, magnetic levitation culture, solid-scaffold-in-well culture, hydrogells-in-well culture, hydrogells-on-micropillar culture, hydrogells-in-microchannel culture, hang-in-drop culture, U-shape-well culture, and V-shape well culture.

In an embodiment of the present disclosure, the 3D cell culture may be performed by pellet culture. The pellet culture may be effective in maintaining a phenotype of the chondrocytes, and may be able to provide an extracellular environment similar to the initial cartilage tissue generation environment by easily aggregating cells through centrifugation to induce a cell-to-cell bonding effect.

The term “spheroid” as used herein refers to a cell structure designed in a 3D manner.

In an embodiment of the present disclosure, the MATN-3 protein is related to differentiation and regeneration of cartilage. In this regard, when stem cells are primed with MATN-3, differentiation into chondrocytes may be promoted while hypertrophy and dedifferentiation of chondrocytes may be inhibited. When culturing stem cells in a medium supplemented with the MATN-3 protein at a concentration in a range of about 5 ng/ml to about 50 ng/ml, the MATN-3 priming may result the best effect. Here, the concentration of the MATN-3 protein may be in a range of about 5 ng/ml to about 45 ng/ml, about 5 ng/ml to about 40 ng/ml, about 5 ng/ml to about 35 ng/ml, about 5 ng/ml to about 30 ng/ml, about 5 ng/ml to about 25 ng/ml, about 5 ng/ml to about 23 ng/ml, about 5 ng/ml to about 20 ng/ml, about 5 ng/ml to about 18 ng/ml, about 5 ng/ml to about 15 ng/ml, about 5 ng/ml to about 13 ng/ml, about 5 ng/ml to about 12 ng/ml, about 7 ng/ml to about 40 ng/ml, about 7 ng/ml to about 35 ng/ml, about 7 ng/ml to about 30 ng/ml, about 7 ng/ml to about 25 ng/ml, about 7 ng/ml to about 20 ng/ml, about 7 ng/ml to about 18 ng/ml, about 7 ng/ml to about 15 ng/ml, about 7 ng/ml to about 13 ng/ml, or about 5 ng/ml to about 10 ng/ml. In an embodiment of the present disclosure, the concentration of the MATN-3 protein may be in a range of about 5 ng/ml to about 15 ng/ml. In one or more embodiments of the present disclosure, the concentration of the MATN-3 protein may be about 10 ng/ml.

In addition, when culturing stem cells for about 80 hours to about 130 hours, the MATN-3 priming may result the best effect. Here, the culturing time period may be in a range of about 80 hours to about 130 hours, about 80 hours to about 130 hours, about 80 hours to about 125 hours, about 80 hours to about 125 hours, about 90 hours to about 130 hours, about 90 hours to about 130 hours, about 90 hours to about 125 hours, about 90 hours to about 125 hours, about 100 hours to about 125 hours, about 100 hours to about 125 hours, about 110 hours to about 130 hours, or about 115 hours to about 125 hours. In an embodiment of the present disclosure, the culturing time period may be in a range of about 110 hours to about 130 hours, and in one or more embodiments of the present disclosure, the culturing time period may be about 120 hours.

In addition, the 3D cell culture of the present disclosure may include culturing cells in a range of about 50 cells per microwell to about 500 cells per microwell. Here, the number of cells may be in a range about 50 cells per microwell to about 450 cells per microwell, about 50 cells per microwell to about 400 cells per microwell, about 50 cells per microwell to about 430 cells per microwell, about 50 cells per microwell to about 400 cells per microwell, about 50 cells per microwell to about 430 cells per microwell, about 50 cells per microwell to about 380 cells per microwell, about 50 cells per microwell to about 350 cells per microwell, about 50 cells per microwell to about 330 cells per microwell, about 50 cells per microwell to about 300 cells per microwell, about 50 cells per microwell to about 280 cells per microwell, about 50 cells per microwell to about 250 cells per microwell, about 50 cells per microwell to about 220 cells per microwell, about 50 cells per microwell to about 200 cells per microwell, about 50 cells per microwell to about 180 cells per microwell, about 50 cells per microwell to about 150 cells per microwell, about 50 cells per microwell to about 140 cells per microwell, about 50 cells per microwell to about 130 cells per microwell, about 60 cells per microwell to about 150 cells per microwell, about 70 cells per microwell to about 150 cells per microwell, about 80 cells per microwell to about 150 cells per microwell, about 90 cells per microwell to about 150 cells, or about 100 cells per microwell to about 150 cells per microwell. In an embodiment of the present disclosure, the number of cells may be in a range of about 80 cells per microwell to about 150 cells per microwell. In one or more embodiments of the present disclosure, the number of cells may be about 125 cells per microwell.

A stem cell spheroid may have an effect of inducing differentiation of adult stem cells into chondrocytes. Furthermore, the stem cell spheroid may have an effect of inhibiting hypertrophy and dedifferentiation of chondrocytes. In addition, when co-cultured with nucleus pulposus cells collected from a patient, the stem cell spheroid may have an effect of regenerating the nucleus pulposus cells. Furthermore, the recovery of extracellular matrix components is also confirmed. When the stem cell spheroid is injected into a cartilage tissue, such as intervertebral disc, chondrocytes are differentiated and bring an effect of regenerating the chondrocytes.

Another aspect of the present disclosure provides a spheroid prepared by the method. The spheroid may have an effect of inducing differentiation of adult stem cells into chondrocytes. Furthermore, the spheroid may have an effect of inhibiting hypertrophy and dedifferentiation of chondrocytes. In an embodiment of the present disclosure, the spheroid refers to a structure obtained by the 3D cell culture, and when co-cultured with nucleus pulposus cells collected from a patient, the nucleus pulposus cells may be regenerated and the extracellular matrix components may be recovered. When the spheroid is injected into a cartilage tissue, such as an intervertebral disc, it is confirmed that chondrocytes are differentiated and chondrocytes are regenerate.

Another aspect of the present disclosure provides a composition for preventing or treating cartilage disease, the composition including the spheroid prepared by the method. For example, the composition may refer to a composition for preventing or treating cartilage disease, the composition including a spheroid prepared by steps of: culturing stem cells in a medium supplemented with a MATN-3 protein; and performing 3D cell culture on the cultured stem cells in the medium.

The pharmaceutical composition may further include adult stem cells as active ingredients.

The adult stem cells may be, for example, adipose-derived mesenchymal stem cells.

The MATN-3, the culture, the stem cells, and the spheroid are respectively the same as described above.

The term “cartilage disease” as used herein refers to a cartilage-related disease that requires differentiation and regeneration of cartilage. Examples of the cartilage disease are degenerative intervertebral disc, osteoarthritis, degenerative disc, intervertebral disc herniation, degenerative arthritis, fracture, muscle tissue injury, plantar fasciitis, humeral epicondylitis, calcific tendinitis, fracture nonunion or traumatic joint injury, osteomalacia, cartilage injury, or cartilage defect, but are not limited thereto. In detail, the cartilage disease may include at least one selected from the group consisting of degenerative intervertebral disc, intervertebral disc herniation, osteoarthritis, degenerative arthritis, rheumatic arthritis, osteomalacia, cartilage injury, and cartilage defect.

The pharmaceutical composition of the present disclosure may promote regeneration of cartilage tissues in a joint by inducing specific differentiation of endogenous stem cells or transplanted therapeutic stem cells into chondrocytes. Therefore, unlike the conventional public approaches, such as inflammation control, that treat only rheumatic arthritis in which joint tissues are destroyed due to an inflammatory response caused by an abnormal immune function, a wide range of joint diseases may be treated by using the pharmaceutical composition of the present disclosure. In addition, the pharmaceutical composition of the present disclosure may enable fundament treatment of degenerative intervertebral disc and osteoarthritis.

In addition, when the composition for inducing differentiation into chondrocytes of the present invention is used for treatment of cartilage injury and defect of a subject, the composition may be introduced to the body separately from adult stem cells or simultaneously with adult stem cells. In other words, before or after administration of adult stem cells, or at the same time as administration of adult stem cells, the composition including the MATN-3 protein may be separately administered. Here, the pharmaceutical composition may include a known pharmaceutical carrier suitable for administration of the MATN-3 protein.

When the subject to which the pharmaceutical composition including the MATN-3 protein and adult stem cells is administered is a human, the adult stem cells may preferably be those of a patient to which the pharmaceutical composition is administered.

The therapeutic composition may be directly administered into a joint of a patient according to a method known in the art. In addition, a dose of the adult stem cells may be in a range of about 1×10⁴ cells/kg to about 1×10⁸ cells/kg. However, such a dose may vary depending on weight, age, gender, and lesion severity of a patient. In addition, regarding the administration, it is preferable to adjust the amount of the composition to be administrated and the number of cells in consideration of various related factors including a disease to be treated, disease severity, an administration route, and weight, age, and gender of a patient.

The pharmaceutical composition may further include a known carrier being used in the art for transplantation of stem cells. The pharmaceutical composition of the present disclosure may be applied to a human body by parenteral administration or topical administration. Regarding such administration routs, the active ingredient included in the composition may be suspended or dissolved in a pharmaceutically acceptable carrier according to a conventional method in the art. Here, it is preferable to use a water-soluble carrier.

The chondrocytes subjected to the differentiation induction according to the method of the present disclosure or the pharmaceutical composition of the present disclosure may be used as a cell therapy agent for the treatment of various cartilage diseases. A cell therapy agent may include cells and tissues that are isolated from a human, cultured, and prepared by specialized steps, and may refer to a medicine used for treatment, diagnosis, and prophylaxis purposes. For example, the cell therapy agent may refer to a medicine used for treatment, diagnosis, and prophylaxis purposes, prepared through a series of actions including: in vitro proliferating and screening of living autologous, allogeneic, or xenogeneic cells to restore function of cells or tissues; or changing biological properties of cells in other ways.

The cell therapy agent of the present disclosure may be applied to a cartilage injury part of humans or non-human creatures, such as non-human mammals including cattle, monkeys, birds, cats, mice, rats, hamsters, pigs, dogs, rabbits, sheep, horses, and the like, to promote regeneration (differentiation) of cartilage or treat cartilage injury by injection into a joint.

The cell therapy agent may be directly injected into a joint of a patient according to a known method in the art, or may be transplanted with a scaffold after 3D cell culture. Here, the number of cells to be administrated may be adjusted in consideration of various related factors, such as disease to be treated, disease severity, an administration route, and weight, age, and gender of a patient.

In addition, the composition or the cell therapy agent of the present disclosure may be inoculated first on a scaffold used for cartilage formation, and then applied to a cartilage injury part. For use as the scaffold, various forms including sponge, gel, fiber, and microbead may be used. In particular, a porous structure that can improve a cell adhesion rate and maintain a high rate of surface tension relative to volume be used.

Another aspect of the present disclosure provides a method of preventing, improving, or treating cartilage disease in a subject, the method including administering the pharmaceutical composition for treating or preventing cartilage disease to a subject in an amount effective for prophylaxis or treatment of the cartilage disease. The cartilage disease, the prophylaxis, and the treatment are respectively the same as described above.

Here, the administration may be topical administration or systemic administration. For example, the administration may be oral, rectal, intravenous, nasal, intraperitoneal, subcutaneous, or topical administration. The topical administration may be, for example, directly applied to lesion, or around lesion. The administration may refer to administration of the pharmaceutical composition in an effective amount for the prophylaxis or treatment of the disease. Such an effective amount may be easily selected by a person skilled in the art depending on a disease condition. In addition, the pharmaceutical composition of the present disclosure may be administered by using any device capable of delivering an effective ingredient to a target cell.

Another aspect of the present disclosure provides use of the pharmaceutical composition for treating or preventing cartilage disease to prepare a composition for preventing or treating cartilage disease.

### Advantageous Effects of Disclosure

An aspect of the present disclosure provides a method of preparing a spheroid with set conditions and period of matrilin-3 protein priming on stem cells. Another aspect of the present disclosure provides a spheroid prepared by the method and a composition including the spheroid for treating cartilage disease. When the composition according to the aspect of the present disclosure is used, cartilage regeneration and increase in an extracellular matrix may be promoted, and dedifferentiation and hypertrophy phenomena may be reduced, thereby consequently providing more effective methods for cartilage disease and cartilage tissue regeneration.

### BRIEF DESCRIPTION OF DRAWINGS

FIG. 1 is an image showing a spheroid formation method according to an embodiment and the sequence of spheroid efficacy confirmation.

FIG. 2 is a graph showing increased matrilin-3 mRNA and protein expression levels when adipose-derived stem cells are cultured in a culture medium that induces differentiation into cartilage.

FIG. 3 is a graph showing increase expression levels of collagen 2 and aggrecan, which are markers related to cartilaginification, in stem cells that are cultured after the stem cells are primed with MATN-3.

FIGS. 4A to 4F are graphs showing increased expression levels of collagen 2 and aggrecan, which are markers related to cartilaginification, in a 3D pellet tissue after the stem cells are respectively primed with matrilin-3 at different concentrations.

FIGS. 5A to 5C show experiment results obtained under conditions that a dose of matrilin-3 for priming adipose-derived stem cells is 10 ng/ml, 20 ng/ml, and 50 ng/ml and a period of priming is 1 day (24 hours), 3 days (72 hours), and 5 days (120 hours), wherein G1 refers to a group of stem cells only, G2 refers to a group of stem cells primed with 10 ng/ml of matrilin-3, G3 a group of stem cells primed with 20 ng/ml of matrilin-3, and G4 refers to a group of stem cells primed with 50 ng/ml of matrilin-3. Here, A shows the results obtained by live and dead assay, B shows the results obtained by proliferation assay, and C shows the results obtained by cytokine analysis based on mRNA expression levels of cartilage-related markers, such as SOX9, collagen 2, and aggrecan.

FIG. 6 is an image showing a total of six conditions designed to establish optimal experimental conditions, wherein the six conditions include: a condition of a monolayer of adipose-derived stem cells; a condition of a monolayer of matrilin-3-primed adipose-derived stem cells; a condition of a matrilin-3-primed adipose-derived stem cell spheroid formed of 125 cells per microwell; a condition of a matrilin-3-primed adipose-derived stem cell spheroid formed of 125 cells per microwell; a condition of a matrilin-3-primed adipose-derived stem cell spheroid formed of 250 cells per microwell; and a condition of a matrilin-3-primed adipose-derived stem cell spheroid formed of 500 cells per microwell.

FIG. 7 is a graph showing experiment results obtained to establish optimal experimental conditions when forming a spheroid after priming of adipose-derived stem cells with matrilin-3 for 5 days, wherein G1 refers to a MSC monolayer culture group; G2 refers to a matrilin-3-primed MSC monolayer culture group; G3 refers to a MSC spheroid group; G4 refers to a matrilin-3-primed MSC spheroid group (formed of 125 cells per microwell); and G5 refers to a matrilin-3-primed MSC spheroid group (formed of 250 cells per microwell. FIG. 7A shows results of live and dead assay; FIG. 7B shows mRNA expression levels of p53 and BAX that are apoptosis markers; FIG. 7C shows results of human cytokine array analysis used to determine growth factors and cytokines in conditioned media of Ad-MSCs and primed Ad-MSCs; FIG. 7D is an image showing signal densities normalized as a positive control; and FIG. 7E is an image showing signal densities normalized as a positive control.

FIG. 8 is a graph showing results that cartilage differentiation markers increase and cartilage hypertrophy markers decrease when co-culturing matrilin-3-primed adipose-derived stem cell spheroids and degenerative nucleus pulposus (dNP) cells. FIG. 8A shows mRNA expression levels of SOX9, collagen 2, and aggrecan, which are cartilage differentiation markers, in dNPs; and FIG. 8B shows mRNA expression levels of cartilage hypertrophy markers, such as collagen 10, collagen 1, and MMP13 in dNP (***p<0.001, **p<0.01, * p<0.05).

FIG. 9 is a graph showing results obtained by co-culturing matrilin-3-primed adipose-derived stem cell spheroids and dNP cells. FIG. 9A shows immunofluorescence images of cadherin 2 and fluorescent intensity of the dNP cells, FIG. 9B shows immunofluorescence images of chondroitin sulphate and fluorescent intensity of the dNP cells, and FIG. 9C shows immunofluorescence images of collagen I and fluorescent intensity of the dNP cells (***p<0.001, **p<0.01, * p<0.05).

FIG. 10 shows images depicting a process of preparing a rabbit model with degenerative lumbar intervertebral disc by a retroperitoneal approach, wherein an image on the right shows that an 18-gauge needle is selected for insertion as a result of using various sizes of spinal needles.

FIG. 11 is an image showing T MRI results to verify regeneration of degenerative lumbar intervertebral disc, wherein a dotted line arrow indicates a defected site, and a solid line arrow indicates a normal site in contrast to the defected site. From left to right, spinal MRI analysis images of a group of non-primed cells, a group to which only stem cells are administered, a group to which matrilin-3-primed stem cells are administered, a group to which stem cell spheroids are administered, and a group to which matrilin-3-primed stem cell spheroids are administered are shown.

FIG. 12 is a graph showing the results of Masson's trichrome staining on tissues to clarify the MRI results in more detail. From top to bottom, histological analysis results of a group of non-primed cells, a group to which only stem cells are administered, a group to which matrilin-3-primed stem cells are administered, and a group to which stem cell spheroids are administered are shown. G5 at the bottom shows a histological analysis result of a group to which adipose-derived stem cell spheroids are administered.

FIG. 13 shows a schematic diagram depicting experiment results.

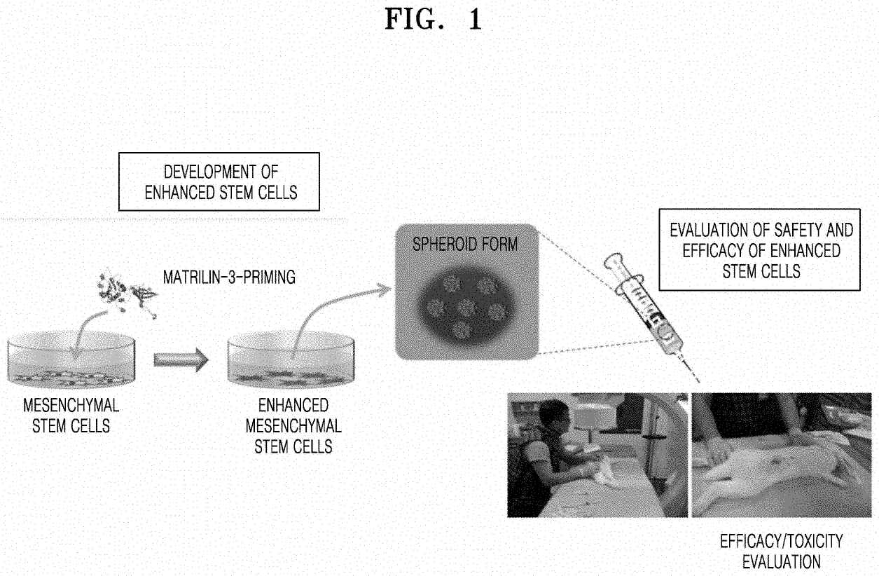

### MODE OF DISCLOSURE

Hereinafter, the present disclosure will be described in more detail through Examples. However, these Examples are for illustrative purposes of the present disclosure only, and the scope of the present disclosure is not limited thereto.

### Example 1. Confirmation of Effect of Matrilin-3 (MATN-3) Protein on Cartilage Differentiation of Human Adipose-Derived Stem Cells

To confirm effect of MATN-3 protein on cartilage differentiation of human adipose-derived stem cells, the inventors of the present disclosure used human adipose-derived stem cells for MATN-3 priming.

1.1. Isolation of Human Adipose-Derived Stem Cells

To isolate human adipose-derived stem cells, adipose tissues to be removed and discarded by liposuction were collected and washed with phosphate buffered saline (PBS). The washed adipose tissues were treated with 1.5 mg/ml of collagenase, and then filtered through a 70 μm-scale nylon mesh. Red blood cells were removed from the filtrate by using a hemolysis buffer solution (0.15 M NH₄Cl, 10 mM KHCO₃, 0.1 mM EDTA), and a washing process was performed thereon twice by using PBS, so as to obtain adipose-derived stem cells. The adipose-derived mesenchymal stem cells thus obtained were seeded at a concentration of 1×10⁴/cm² on a culture plate containing a DMEM medium supplemented with 10% fetal bovine serum (FBS) and 1% antibacterial agent, and cultured with 5% CO₂ at 37° C. When the cells covered about 80% of the bottom area of the culture plate, the cells attached to the bottom surface were isolated from the culture plate by using trypsin/EDTA. Then, the isolated cells were centrifuged at 1,200 rpm for 5 minutes, suspended again in the same medium, and subcultured in the same manner three times. Accordingly, passage 3 cells were used for next experiments.

1.2. Confirmation of Increased Expression Level of MATN-3 by Chondrogenesis Induction of Stem Cells

A total of 2×10⁵ adipose-derived stem cells were collected in a 15 mL falcon tube, and centrifuged at 120 rpm for 3 minutes. A culture containing pellets of adipose-derived stem cells obtained by the centrifugation was cultured with 5% CO₂ at 37° C. The pellets were divided into two groups, and one group was treated with a serum free (SF) medium, and the other group was treated with chondrogenic (CM) medium. The CF medium was supplemented with DMEM-high glucose, 10% fetal bovine serum, 100× insulin-transferrin-selenium (ITS), 50 ng/ml of ascorbic acid, 100-nM dexamethasone, 1× penicillin and streptomycin, and 10-ng/ml of TGF-β. Each medium was changed every 3 days with a fresh medium, and after 21 days of the culture, the increase in the MATN-3 expression of the adipose-derived stem cells cultured in each of the SF medium and the CF medium was compared.

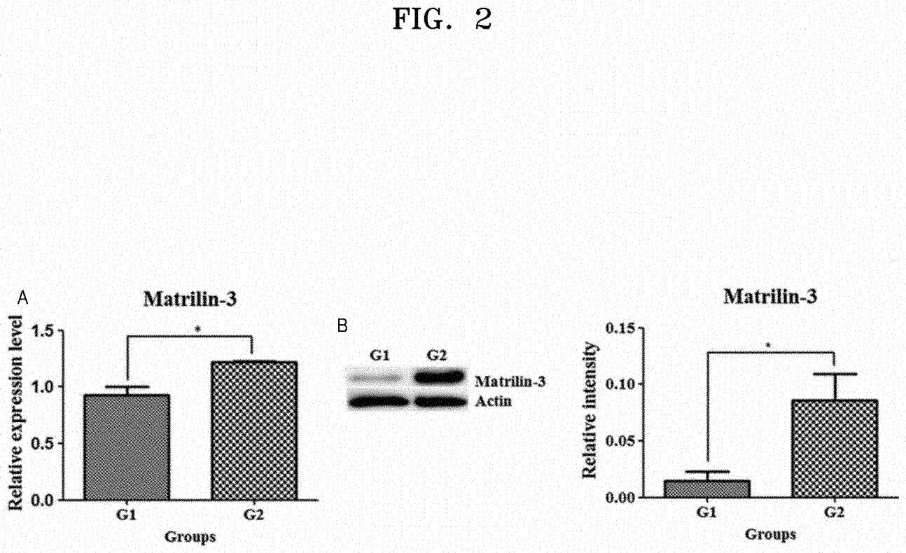

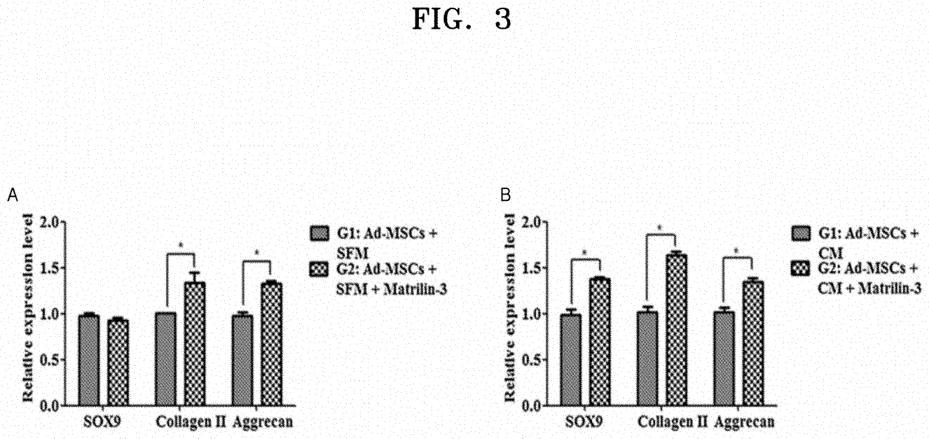

Consequently, it was confirmed that the MATN-3 mRNA and protein expression levels increased by the induction of chondrogenesis of the stem cells (see FIG. 2). Furthermore, it was also confirmed that, when the adipose-derived stem cells were cultured in a culture medium for inducing cartilage differentiation, the MATN-3 secretion was increased (see FIG. 3). Accordingly, the inventors of the present disclosure could confirm that MATN-3 was a protein related to the cartilage differentiation.

1.3. Analysis of Expression of Cartilage-Related Genes

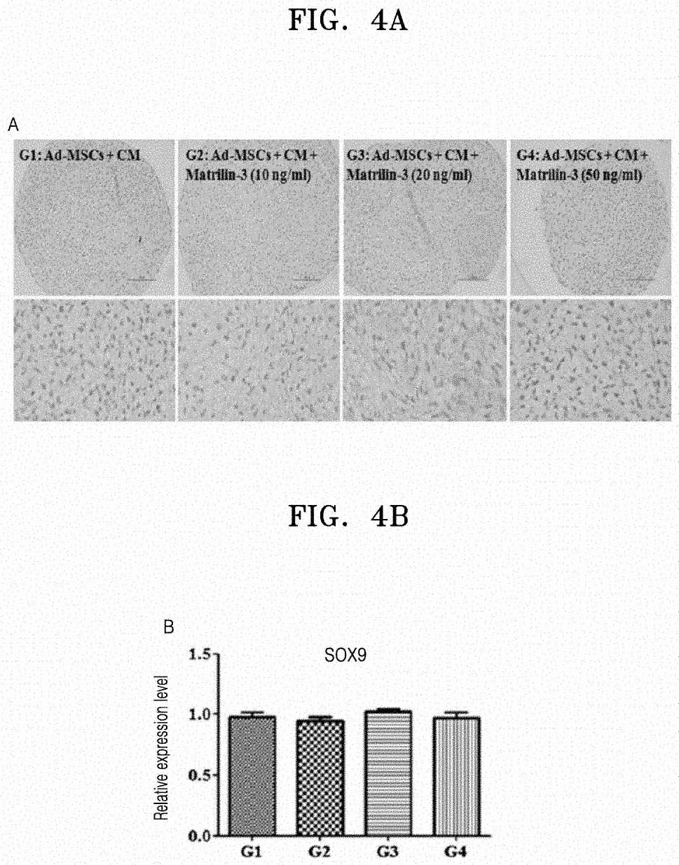

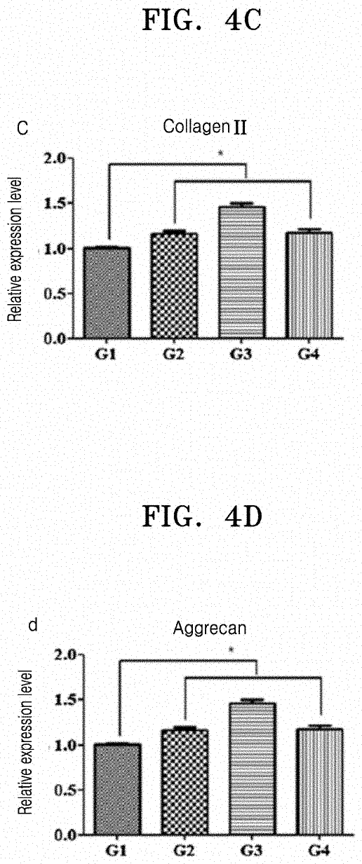

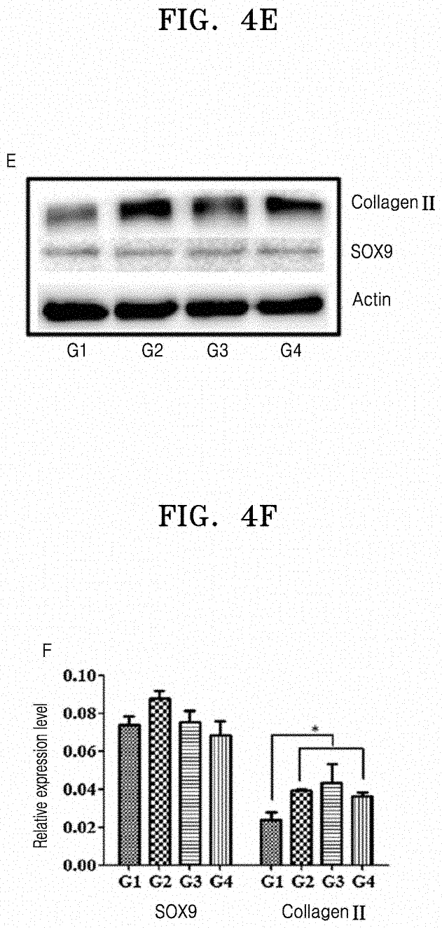

To confirmed effects of the MATN-3 protein priming on the expression of cartilage genes in the adipose-derived stem cells, passage 3 cells were collected, divided into groups for every 2×10⁵ adipose-derived stem cells, and centrifuged at 1,200 rpm for 3 minutes. The resultant cells were cultured for 24 hours in the form of pellets in a FBS-free DMEM medium as being divided into a group with MATN-3 protein and a group without MATN-3 protein. After 24 hours of the culture, for quantitative analysis of the obtained cells after removing the medium, quantitative real-time polymerase chain reaction (qRT-PCR) was performed to measure RNA expression of the cartilage-related genes. That is, the obtained cell pellets were washed with PBS three times, and collected by using trypsin/EDTA. Then, RNA of the cells was extracted according to a TRIzol method (Life Technologies, Inc. Grand Island, N.Y.). 1 μg of the extracted RNA was used to synthesize cDNA by using a cDNA synthesis kit (AB biosystems), and qRT-PCR was performed thereon by using Master SYBR green (AB biosystems). Cell normalization was performed by using glyceraldehyde 3-phosphate dehydrogenase (GAPDH), and primer sets and respective cartilage-related gene markers used in the qRT-PCR are as shown in Table 1. Accordingly, the inventors of the present disclosure were able to confirm that the MATN-3 protein priming increased the expression of cartilage-related genes (see FIG. 4).

TABLE 1

| | | Accession | Amplicon |
|---|---|---|---|
| Gene | Primer sequence | number | (bp) |
| 18S | 5′-GTA ACC CGT TGA | NR_003286.2 | 151 |
| | ACC CCA TT-3′5′-CCA | | |
| | TCC AAT CGG TAG TAG | | |
| | CG-3′ | | |
| SOX9 | 5′- GTA CCC GCA CTT | NM_000346.3 | 74 |
| | GCA CAA C-3′5′- TCT | | |
| | CGC TCT CGT TCA GAA | | |
| | GTC-3′ | | |
| Collagen | 5′- GGGAGTAATGCAAGG | NM_001844.4 | 175 |
| 2a | ACCA - 3′5′-ATCATCA | | |
| | CCAGGCTTTCCAG -3′ | | |
| Aggrecan | 5′- GCC TGC GCT CCA | NM_013227.3 | 104 |
| | ATG ACT - 3′5′-ATG | | |
| | GAA CAC GAT GCC TTT | | |
| | CAC -3′ | | |

### Example 2. Preparation Process of MATN-3-Primed Adipose-Derived Stem Cell Spheroid and Confirmation of Regeneration Effects of the MATN-3-Primed Adipose-Derived Stem Cell Spheroid

2.1. Determination of MATN-3 Dose and Period with Respect to Adipose-Derived Stem.

As stem cells for priming with MATN-3, human adipose-derived stem cells were used. First, cells were not fed with nutrients for 12 hours, and subjected to a priming process. Here, determining appropriate MATN-3 concentration and priming period was a significant concern. In the present experiment, to establish appropriate MATN-3 concentration for priming of the adipose-derived stem cells, MATN-3 concentration conditions were set to 10 ng, 20 ng, and 50 ng, and priming period conditions were was set to 1 day, 3 days, and 5 days. Afterwards, the cells were collected.

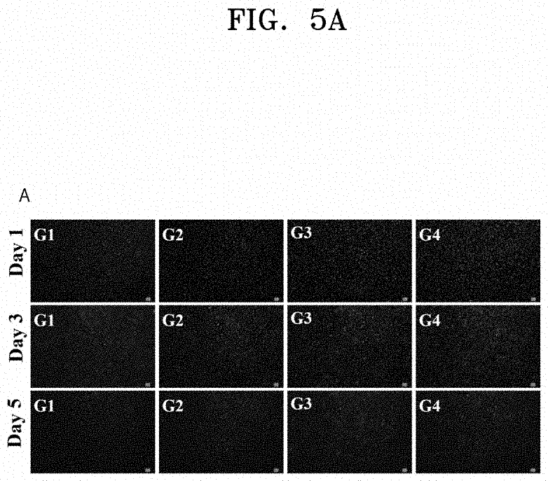

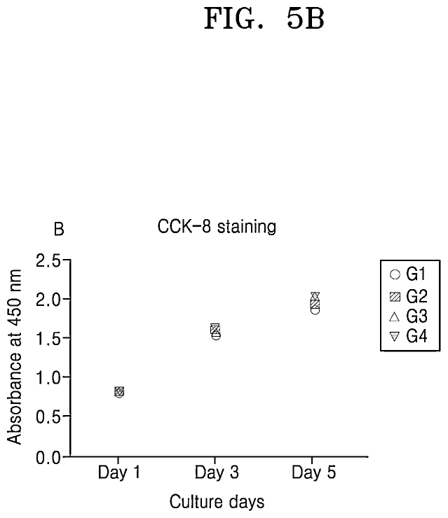

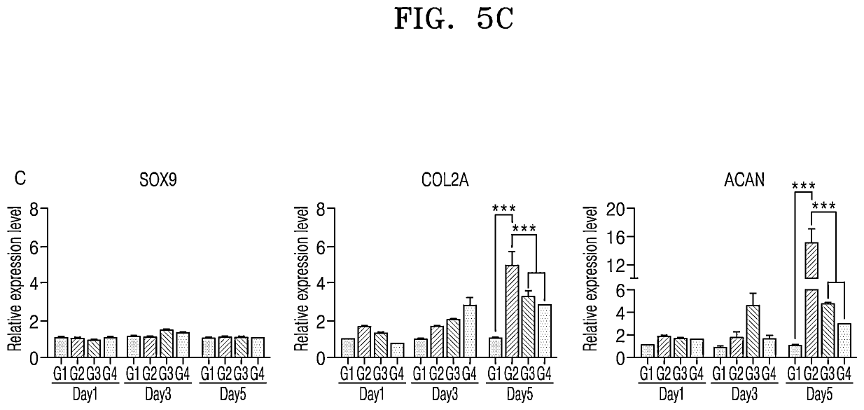

To determine whether the MATN-3 dose and the priming period were optimal conditions, the inventors of the present disclosure transported the cells to a 6-well plate (EZSPHERE). After 48 hours, cytokine array was performed thereon. For the cytokine array, a film customized by using C-series of RayBiotech Ltd based on the sandwich immunoassay principles was used. The customized film was designed as shown in Table 2. Regarding the results visualized in digital images after the measurement, the fold change was calculated for each protein, and the expression levels of SOX9, collagen 2, and aggrecan were compared with one another. As a result, it was confirmed that, when the human adipose-derived stem cells were isolated, the MATN-3 concentration was 10 ng/ml, and the MATN-3 priming period was 5 days, the greatest gene content and the highest synthesis degree were resulted (see FIG. 5B), and that the cartilage differentiation markers (e.g., collagen 2 and aggrecan) were expressed to the maximum. Consequently, it was confirmed that the concentration and period conditions above were optimal conditions to exhibit maximum effects on the cartilage differentiation by MATN-3 priming to the stem cells.

TABLE 2

| | A | B | C | D | E | F | G | H | I | J | K | L |
|---|---|---|---|---|---|---|---|---|---|---|---|---|
| 1 | POS | POS | NEG | NEG | TGF-β1 | TGF-β2 | TGF-β3 | SDF-1 | bFGF | EGF | G-CSF | RANTES |
| | | | | | | | | | | | | (CCL5) |
| 2 | POS | POS | NEG | NEG | TGF-β1 | TGF-β2 | TGF-β3 | SDF-1 | bFGF | EGF | G-CSF | RANTES |
| | | | | | | | | | | | | (CCL5) |
| 3 | IL-11 | IL-1β | IL-6 | MMP-1 | MMP-9 | TNF-α | TIMP-1 | TIMP-2 | HGF | VEGF | IGF-1 | GDF-15 |
| 4 | IL-11 | IL-1β | IL-6 | MMP-1 | MMP-9 | TNF-α | TIMP-1 | TIMP-2 | HGF | VEGF | IGF-1 | GDF-15 |
| 5 | BMP-2 | BMP-7 | BMP-9 | Adipsin | MMP-13 | Activin-A | IL-4 | IL-10 | Matrilin-3 | IL-1ra | BLANK | POS |
| 6 | BMP-2 | BMP-7 | BMP-9 | Adipsin | MMP-13 | Activin-A | IL-4 | IL-10 | Matrilin-3 | IL-1ra | BLANK | POS |

2.2. Standardization of Spheroid Formation Conditions after MATN-3 Priming

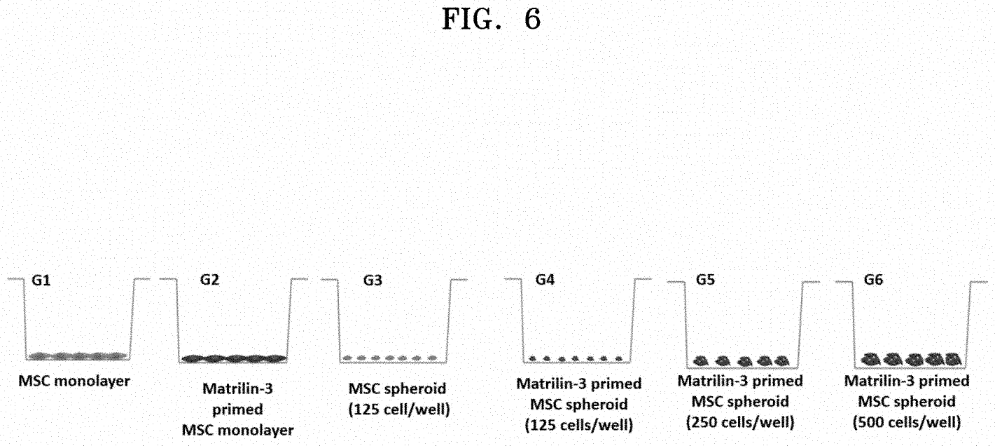

Next, the inventors of the present disclosure carried out an experiment to determine the optimal culture environment conditions for the spheroid formation after MATN-3 priming. A total of six culture conditions were set up as follows: a condition of adipose-derived stem cell monolayer; a condition of MATN-3-primed adipose-derived stem cell monolayer; a condition of adipose-derived stem cell spheroids formed of 125 cells per microwell; a condition of MATN-3-primed adipose-derived stem cell spheroids formed of 125 cells per microwell; a condition of MATN-3-primed adipose-derived stem cell spheroids formed of 250 cells per microwell; and a condition of MATN-3-primed adipose-derived stem cell spheroids formed of 500 cells per microwell (see FIG. 6).

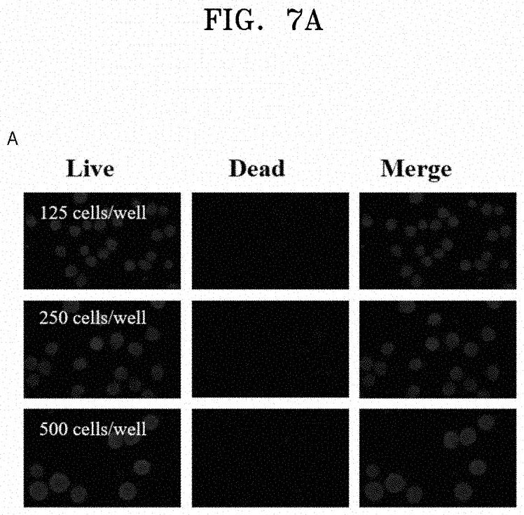

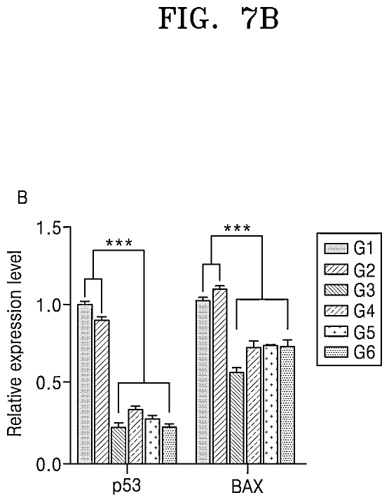

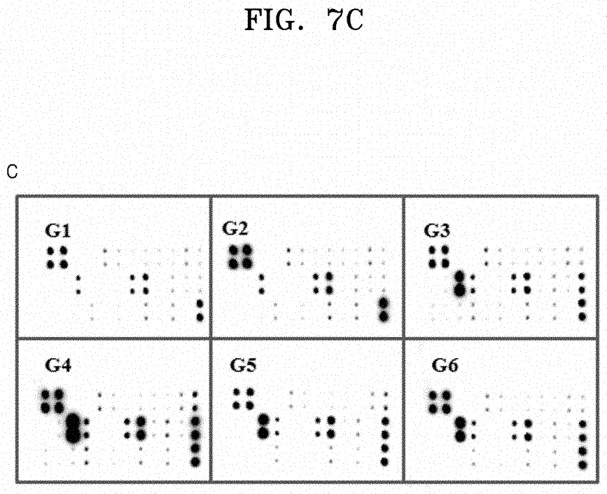

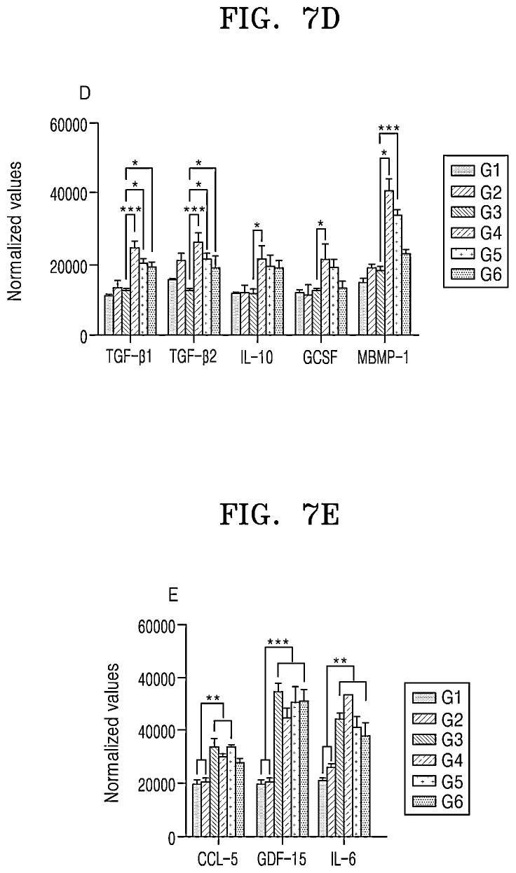

The human adipose-derived stem cells were placed in a cell culture plate, and cultured with 5% CO₂ at 37° C. After 12 hours of the culture, the culture medium was changed to a serum starvation medium (supplemented with DMEM-LG and 1× penicillin and streptomycin), and cultured for 12 hours in a CO₂ incubator. After 12 hours of serum starvation, the culture medium was supplemented with 10 ng/mL of MATN-3. The culture medium was changed every 24 hours with fresh MATN-3 supplement for 5 days. Next, the stem cells were transported to a 6-well plate (EZSPHERE), and then seeded at a density of 125 cells per microwell. 3 mL of a mixed solution containing 10% fetal bovine serum (FBS) and gentamicin (50 μg/ml) in Dulbecco's modified Eagle's medium (DMEM)-low glucose (LG) was added thereto and cultured for 24 hours in a CO₂ incubator with 5% CO₂ at 37° C. As such, spheroids were formed through this process. Consequently, it was confirmed that, when 125 cells were seeded, apoptosis markers were expressed the least (see FIG. 7B) while cytokines related to the cartilage formation were secreted the most (see FIGS. 7C to 7E).

As a result of comprehensive analysis on the concentration conditions, the culture environments, and the culture period for the MATN-3 priming to the adipose-derived stem cells, it was confirmed that the optimized culture environment system was established and the most excellent effects of cartilage formation were exhibited when the spheroids were formed under conditions that the MATN-3 concentration was 10 ng/ml, the culture period was 5 days, and 125 cells per microwell were seeded.

### Example 3. Analysis of Regenerative Effect of MATN-3-Primed Spheroid on Degenerated Nucleus Pulposus Cells

3.1. Co-Culture of MATN-3-Primed Spheroids and Nucleus Pulposus Cells

To confirm regenerative effect of the spheroids formed by the method disclosed herein on cartilage nucleus pulposus cells, the inventors of the present disclosure carried out the following experiments. First, spheroids were formed as described in Example 2.2. Then, nucleus pulposus cells were collected for 10 days from a patient undergoing surgery for degenerative lumbar intervertebral disc. After obtaining the approval from Institutional Review Board (IRB) in a hospital, informed consent was obtained in advance from a patient undergoing discectomy for cervical or lumbar herniation of nucleus pulposus. The nucleus pulposus and annulus fibrosus were isolated from the intervertebral disc obtained during surgery, and the nucleus pulposus cells were isolated from the nucleus pulposus. To isolate the nucleus pulposus cells from the nucleus pulposus, disc tissues were washed three times, each for 15 minutes, by using Dulbecco's phosphate-buffered saline (DPBS; Hyclone Laboratories) containing 1% penicillin and streptomycin (Gibco, BRL, USA). The tissue samples were digested with 0.05% (w/v) type 2 collagenase (Sigma Aldrich, St Luis, N.J., USA) for 6 hours. The digested mixture was transported to a cell strainer (40 μm pore size, Becton Dickinson, Franklin Lakes, N.J., USA), centrifuged at 1,000 rpm for 5 minutes, and washed with HBSS twice to remove the remaining collagenase. The cells were suspended in DMEM-LG supplemented with 10% FBS, 0.1 mg/ml of streptomycin, and 100 μg/ml of penicillin, and cultured until the cells were 85% confluent. The nucleus pulposus cells and the MATN-3-primed spheroids were used for co-culture experiments.

3.2. Confirmation of Regenerative Effect of MATN-3-Primed Spheroid on Nucleus Pulposus Cell

The inventors of the present disclosure carried out the co-culture method of Example 3.1, and confirmed the expression of the cartilage-related markers for quantitative analysis of cells. The quantitative analysis was carried out by measuring RNA levels of the cartilage-related markers. RNA was extracted by using a TRIzol kit (ThermoFisher Scientific, Inc., Waltham, Mass., USA). Then, complementary DNA was subsequently prepared by using 0.5 μg of RNA with the Primescript RT reagent kit (Takara Bio Inc, Japan). RT-PCR amplification was performed on the complementary DNA by using the StepOnePlus Real Time PCR System. After the amplification, relative mRNA expression levels were calculated for each target gene. Such calculation used a 2_{-ΔCt} method with the expression level of 18-S as an internal control. Target primers used for real-time (RT)-PCR analysis are shown in Table 3.

TABLE 3

| | | Accession | Amplicon |
|---|---|---|---|
| Gene | Primer sequence | number | (bp) |
| 18S | 5′- GTA ACC CGT | NR_003286.2 | 151 |
| | TGA ACC CCA TT- | | |
| | 3′5′-CCA TCC AAT | | |
| | CGG TAG TAG CG- | | |
| | 3′ | | |
| SOX9 | 5′- GTA CCC GCA | NM_000346.3 | 74 |
| | CTT GCA CAA C- | | |
| | 3′5′- TCT CGC | | |
| | TCT CGT TCA GAA | | |
| | GTC -3′ | | |
| Collagen | 5′- GGGAGTAATGCA | NM_001844.4 | 175 |
| 2a | AGGACCA - 3′5′ - | | |
| | ATCATCACCAGGCTTT | | |
| | CCAG - 3′ | | |
| Aggrecan | 5′- GCC TGC GCT | NM_013227.3 | 104 |
| | CCA ATG ACT - 3′ | | |
| | 5′- ATG GAA CAC | | |
| | GAT GCC TTT | | |
| | CAC - 3′ | | |
| Collagen | 5′- CCC CTG GAA | NM_000088.3 | 148 |
| A1 | AGA ATG GAG ATG- | | |
| | 3′5 -TCC AAA CCA | | |
| | CTG AAA CCT | | |
| | CTG - 3′ | | |
| MMP13 | 5′- TCA CCA ATT | NM_002427.3 | 95 |
| | CCT GGG AAG | | |
| | TCT - 3′5′- TCA | | |
| | GGA AAC CAG GTC | | |
| | TGG AG -3′ | | |
| Collagen | 5′- ACG CTG AAC | NM_000493.3 | 101 |
| 10 | GAT ACC AAA TG - | | |
| | 3′5′- TGC TAT | | |
| | ACC TTT ACT CTT | | |
| | TAT GGT GTA- 3′ | | |
| p53 | 5′- GGCCCACTTCAC | NM_000546 | 156 |
| | CGTACTAA - 3′5′- | | |
| | GTGGTTTCAAGGCCAG | | |
| | ATGT - 3′ | | |
| BAX | 5′- TTTGCTTCAGGG | NM_001291428.1 | 246 |
| | TTTCATCC - 3′5′- | | |
| | CAGTTGAAGTTGCCGT | | |
| | CAGA - 3′ | | |

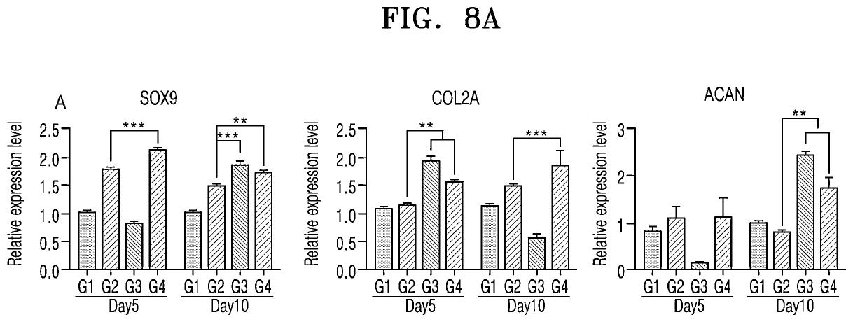

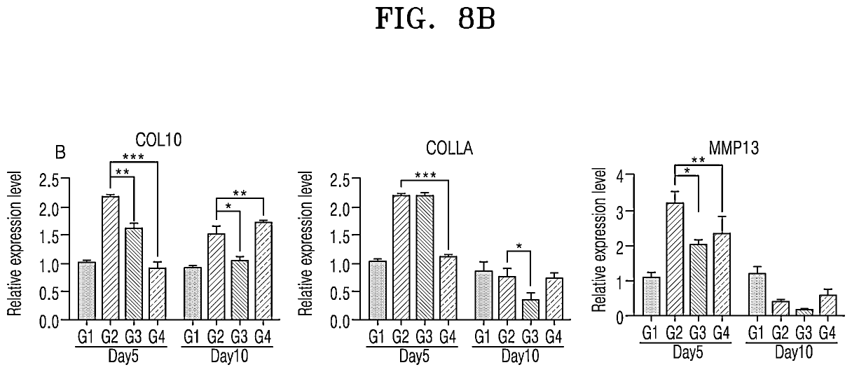

As a result, it was confirmed that, when the MATN-3-primed adipose-derived stem cell spheroids were co-cultured with the degenerated nucleus pulposus cells, the expression levels of the cartilage differentiation markers, i.e., SOX9, Collagen 2, and Aggrecan, were increased, whereas the expression levels of cartilage hypertrophy marker, i.e., Collagen 10 and Collagen 1, were decreased (see FIG. 8). Furthermore, immunohistochemical staining was performed to confirm the effect of the MATN-3-primed adipose-derived stem cell spheroids on the degenerated nucleus pulposus cells. For the immunohistochemical staining, the degenerative nucleus pulposus cells were co-cultured for 10 days with MATN-3-primed adipose-derived stem cell spheroids and MATN-3-non-primed adipose-derived stem cell spheroids. These cells were fixed for 10 minutes with 4% paraformaldehyde at room temperature, washed three times with 1×PBS, and permeabilized with 0.5% Triton-X for 10 minutes. The resultant cells were washed with PBS and blocked for 45 minutes in blocking buffer (5% BSA and 0.5% Tween-20 in 1×PBS) containing 10% normal goat serum (5% BSA and 0.5% Tween-20 in 1×PBS) at room temperature. For immunostaining, the cells were cultured overnight at 4° C. with cadherin-2 (1:200, Abcam), chondroitin sulfate (1:100, Abcam), and collagen 1 (1:200, Abcam). Then, the cells were incubated at a constant temperature with secondary antibodies, i.e., goat anti-rabbit Alexa Fluor® 568 and goat anti-mouse Alexa Fluor® 488 (Abcam), for 1 hour at room temperature. The cells were counter-stained with DAPI (Vector Laboratories, Burlingame, Calif., USA), and images were obtained by using Cytation 3 Cell Imaging Multi-mode Reader (Biotek Instruments, Inc., Winooski, Vt., USA). The detected autofluorescence intensities were used to analyze the expression.

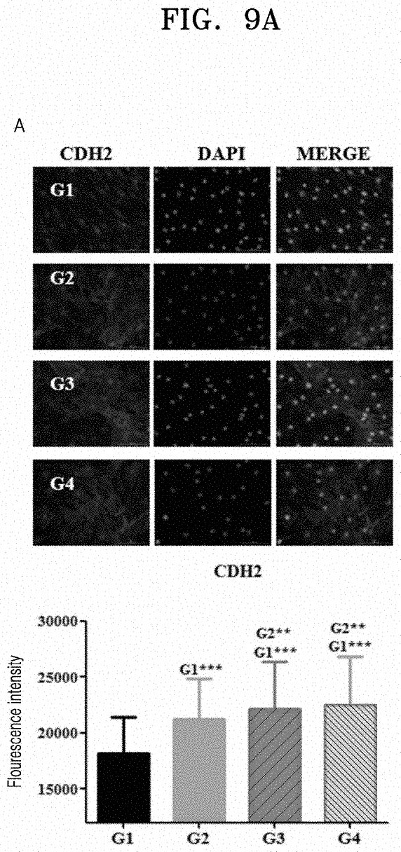

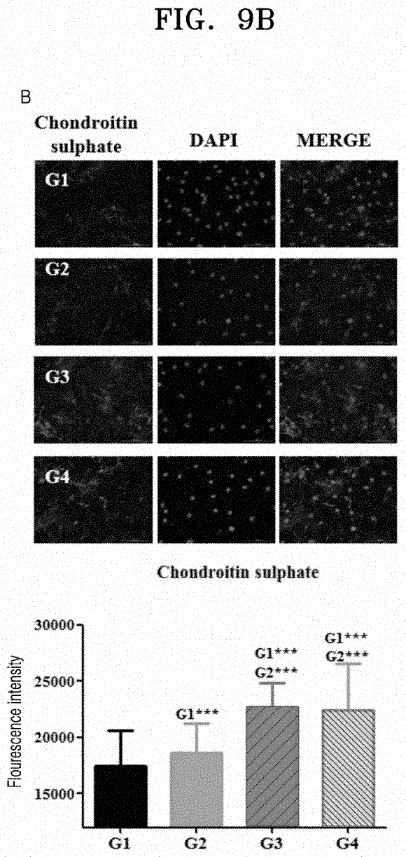

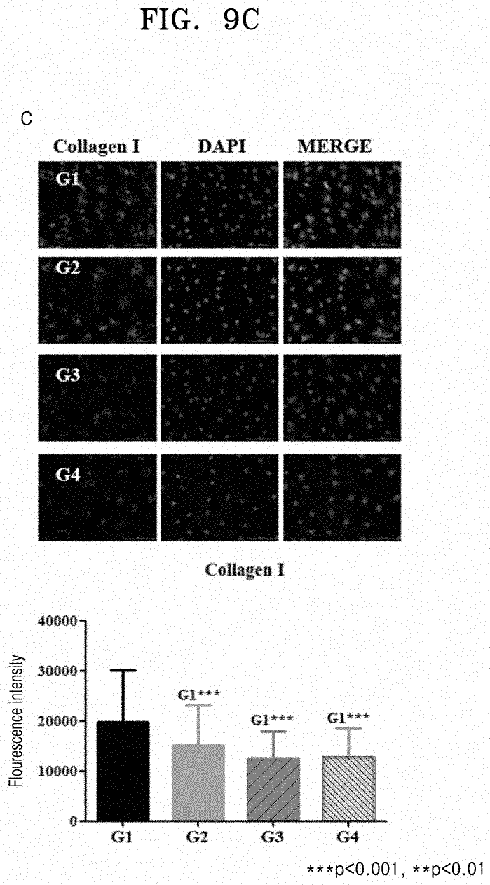

When the MATN-3-primed adipose-derived stem cell spheroids were co-cultured with the degenerated nucleus pulposus cells, the regeneration of the degenerated nucleus pulposus cells and the recovery of the extracellular matrix components were observed. In particular, it was confirmed that, among the extracellular matrix components, the expression levels of cadherin 2 and chondroitin sulfate increased (see FIGS. 9A and 9B), whereas the expression level of collagen 1 decreased (FIG. 9C).

### Example 4. Confirmation of Cartilage Regeneration Effect of MATNn-3-Primed Adipose-Derived Stem Cell Spheroids in Rabbit Model with Degenerative Disc Disease

4.1 Preparation of Rabbit Model and Transplantation of Therapeutic Material

Effects of the MATN-3-primed adipose-derived stem cell spheroids on disc regeneration in an animal model with degenerative disc disease were evaluated.

Here, a total of five classified groups were used as follows: G1 refers to a group to which only adipose-derived stem cells were injected; G2 refers to a group to which MATN-3-primed adipose-derived stem cells were injected; G3 refers to a group to which adipose-derived stem cell spheroids were injected; refers to a group to which MATN-3-primed adipose-derived stem cell spheroids were injected; and G5 refers to a group to which MATN-3-primed adipose-derived stem cell spheroids were injected. All groups were set to include three female rabbits.

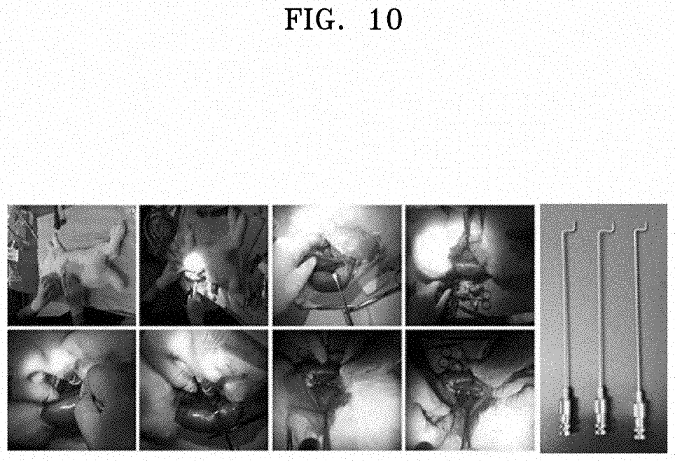

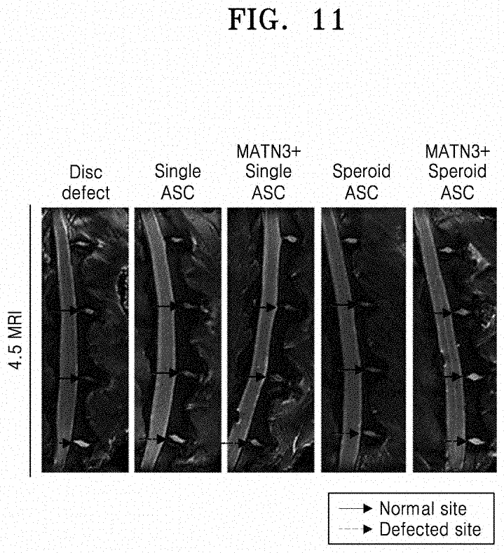

For the preparation of the rabbit model, New Zealand white rabbits (2.5 kg or more in weight) were used as animal models with degenerative lumbar intervertebral disc in accordance with permission from the Institutional Animal Care and Use Committee of CHA University (see FIG. 10). The experiments carried out herein were non-clinical experiments on the intervertebral disc regeneration therapy by using MATN-3-primed stem cell spheroids. For surgery, the rabbits were anesthetized by intramuscular injection of a mixture of 15 mg/kg of Zoletil and 5 mg/kg of Rompun. The incision was made after anesthesia, and the lumbar disc was directly exposed through a retroperitoneal approach after the incision. The procedure was performed on L3/4 discs, L4/5 discs, and L5/6 discs. In detail, the intervertebral discs between the L3/4 discs, the L4/5 discs, and the L5/6 discs were exposed. Then, an 18-gauge spin needle was inserted to inflict damage to the intervertebral discs. At the time of induction of degenerative lumbar intervertebral discs, a therapeutic substance was inserted into each experimental group. Regarding the route of administration, to induce degenerative lumbar intervertebral disc, the intervertebral disc of the lumbar number was exposed through a skin incision on the opposite side of the surgery site, a therapeutic material was directed injected. The administration period was 2 weeks after model the preparation of the disc model, and the number of administration was a single administration. The administration method was to inject 26-gauge spinal needle to death of 5 mm after anesthesia. No immunosuppressants were administered at the time of administration. At 12 weeks after the first insertion of the therapeutic material, MRI and histological examination were performed for efficacy evaluation.

4.2. Confirmation of MRI Results

In the present experiment, MRI was performed to evaluate the degree of degenerative changes in the intervertebral discs. Among the prepared rabbit models, rabbits graded as Pfirrmann grade 3, which represents the standard of degeneration, were used. In the present experiments, as imaging index, T2-weighted images through MRI (wherein time to repetition of 2,000 ms and time to echo of 120 ms) was used. MRI scans of rabbit models in five groups were performed. As a result, it was confirmed that the regeneration of the degenerative lumbar intervertebral disc was confirmed in all groups including a Sham group, an adipose-derived cell injection group, a matrilin-3-primed adipose-derived cell injection group, and a matrilin-3-primed adipose-derived cell spheroid. However, regarding the degree of the effect, it was confirmed that the matrilin-3-primed adipose-derived spheroid injection group of the present disclosure showed the highest signal intensity level and the greatest regenerative effect as compared with the other groups. As a result, the effect of the matrilin-3-primed adipose-derived spheroids on the degenerative lumbar intervertebral disc was confirmed. It addition, it was also confirmed that the effect above may be obtained superior to that of the matrilin-3-primed adipose-derived stem cells.

4.3. Confirmation of Histological Analysis Results

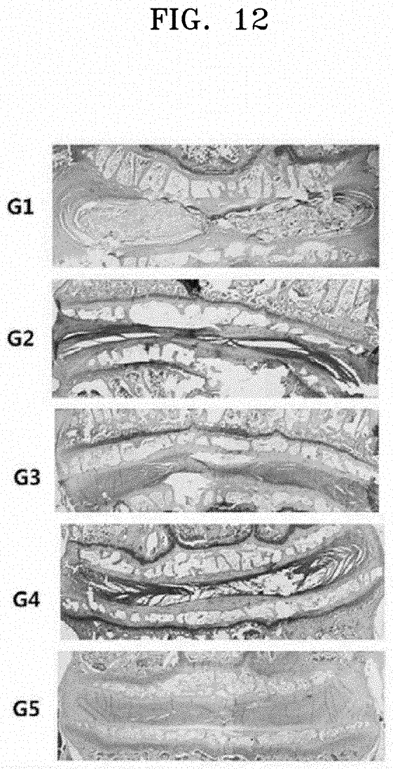

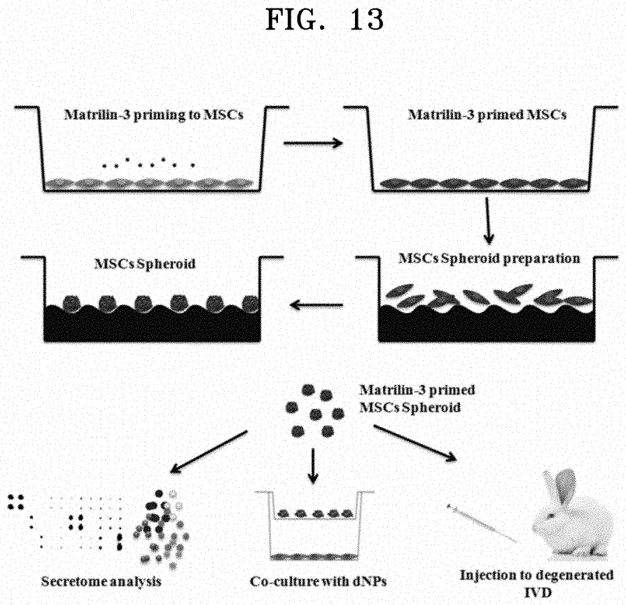

To clarify the MRI results in more detail, tissue staining was performed by Masson's trichrome method. The staining was carried out as follows: cell nucleus was stained in a Weigert iron hematoxylin solution for 10 minutes, cytoplasm and muscles were stained in a Biebrich scarlet-acid fuchsin solution for 15 minutes, and collagen fibers were stained in 2% Aniline blue solution for 3 minutes. After performing the staining, the results were observed under a microscope. As a result, it was confirmed that the group to which the matrilin-3-primed adipose-derived stem cell spheroids were injected showed better regeneration of intervertebral discs than other groups. In addition, it was confirmed that the matrilin-3-primed adipose-derived stem cell spheroids exhibited better effects than the matrilin-3-primed stem cells (see FIG. 12).

1. A method of preparing a spheroid of stem cells, the method comprising:
culturing stem cells in a medium supplemented with matrilin-3 (MATN-3) protein; and
performing 3D cell culture on the cultured stem cells.

2. The method of claim 1, wherein the medium supplemented with the MATN-3 protein has a MATN-3 protein concentration in a range of about 5 ng/ml to about 50 ng/ml.

3. The method of claim 1, wherein a period of the culturing of the stem cells in the medium supplemented with the MATN-3 protein is in a range of about 80 hours to about 130 hours.

4. The method of claim 1, wherein the stem cells are derived from a human.

5. The method of claim 1, wherein the stem cells are adipose-derived mesenchymal stem cells.

6. The method of claim 1, wherein the 3D cell culture is selected from the group consisting of pellet culture, static suspension culture, spinner/rotational chamber culture, nano/micro pattern culture, magnetic levitation culture, solid-scaffold-in-well culture, hydrogels-in-well culture, hydrogels-on-micropillar culture, hydrogels-in-microchannel culture, hang-in-drop culture, U-shape-well culture, and V-shape well culture.

7. The method of claim 1, wherein the 3D cell culture is performed in a range of about 50 cells per microwell to about 500 cells per microwell.

8. A stem cell spheroid prepared by the method according to claim 1.

9. A pharmaceutical composition for preventing or treating cartilage disease, comprising the stem cell spheroid prepared by the method according to claim 1.

10. The pharmaceutical composition of claim 9, wherein the pharmaceutical composition promotes differentiation into chondrocytes and inhibits hypertrophy and dedifferentiation of chondrocytes.

11. The pharmaceutical composition of claim 9, wherein the cartilage disease is selected from the group consisting of degenerative intervertebral disc, degenerative disc, intervertebral disc herniation, osteoarthritis, degenerative arthritis, osteomalacia, cartilage injury, and cartilage defect.

12. A method of preventing, improving, or treating obesity or a metabolic disease, the method comprising administering an effective dose of the pharmaceutical composition of claim 9 to an individual in need thereof.

13. Use of the pharmaceutical composition of claim 9 for preparation of a composition for preventing or treating cartilage disease.
